(19) United States
(12) Patent Application Publication
(10) Pub. No.: US 2025/0285719 A1
(43) Pub. Date: Sep. 11, 2025

(54) System and Method for Veterinary Report Generation

(71) Applicant: ROYA VENTURES INC., TORONTO (CA)

(72) Inventor: PARHAM AARABI, TORONTO (CA)

(21) Appl. No.: 19/074,876

(22) Filed: Mar. 10, 2025

Related U.S. Application Data
(60) Provisional application No. 63/563,601, filed on Mar. 11, 2024.

(51) Int. Cl. G16H 15/00; G06F 40/284; G06F 40/40; G16H 10/40; G16H 50/70

(52) U.S. Cl. CPC G16H 15/00; G06F 40/284; G06F 40/40; G16H 10/40; G16H 50/70

(57) ABSTRACT

Disclosed embodiments relate to the field of veterinary care and, more specifically, to automated veterinary report generation. In an embodiment, a computing system comprises: a diagnostics test system whereby biological samples are collected from a patient and analyzed, a result interpretation system, an expert database and case lookup system, a veterinary-specific language model, and a large language model. These components work synergistically to provide comprehensive and personalized veterinary reports based on numerical test results.

### FIELD OF THE INVENTION

The present disclosure relates to computer systems and improvements thereto, including aspects of machine learning and artificial intelligence and to applications for veterinary care and diagnostics.

### BACKGROUND

It is common in veterinary medicine to perform sample collection and analysis of a biological sample from an animal patient to provide (numerical) sample results to assist with disease diagnosis and/or disease or treatment monitoring. It is desired to provide a computer system and method to interpret sample results and prepare an accurate and contextual report in a natural language form, thereby improving computer systems and/or veterinary healthcare.

### SUMMARY

Disclosed embodiments relate to the field of veterinary care and, more specifically, to automated veterinary report generation. In an embodiment, a computing system comprises: a diagnostics test system whereby biological samples are collected from a patient and analyzed, a result interpretation system, an expert database and case lookup system, a veterinary-specific language model, and a large language model. These components work synergistically to provide comprehensive and personalized veterinary reports based on numerical test results.

### BRIEF DESCRIPTION OF THE DRAWINGS

FIG. 1 is a block diagram showing an environment comprising one or more computing devices according to an embodiment herein and in which aspects disclosed herein can be practiced.

FIG. 2 is a flow chart of operations of the environment of FIG. 1, in accordance with an embodiment.

### DESCRIPTION OF THE INVENTION

### Environment:

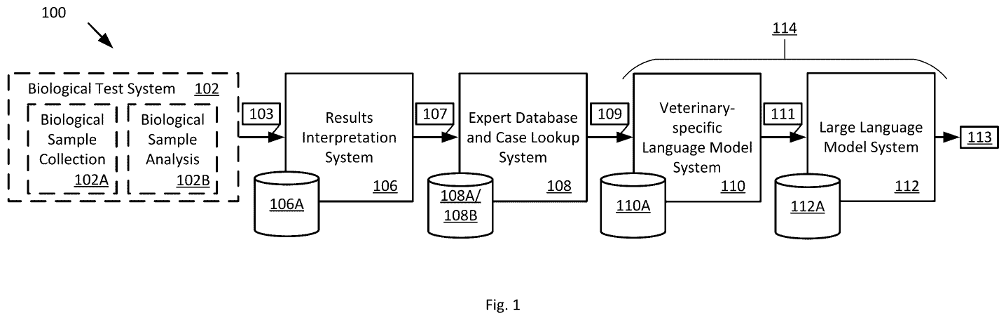

FIG. 1 is a block diagram showing an environment 100 comprising one or more computing devices implementing one or more systems according to an embodiment herein and in which aspects disclosed herein can be practiced. It will be appreciated that while distinct systems are shown for some components, two or more may be implemented by a same computing device.

Environment 100 shows a diagnostic test system 102 having components for biological sample collection 102A and sample analysis 102B. Diagnostic test system 102 is in dotted lines as, in embodiments, one or more features may be performed manually, such as described below. Environment 100 further shows a results interpretation system 106, an expert database and case system 108, a veterinary-specific language model system 110 and a large language model system 112, each with a respective datastore 106A, 108A, 110A and 112A. Respective output 103, 107, 109, 111 and 113 is provided from each system 102-112 as further described.

### Diagnostics Test System:

This system 102 starts with the step of collecting one or more biological samples from a patient in either a manual, semi-automatic, or automatic fashion from a patient. This could include, but is not limited to, the collection of blood, urine, stool sample, or other biological samples. Once the samples are collected, they are analyzed in a manual, semi-automatic, or automatic fashion and interpreted to specific diagnostics results which could be qualitative or quantitative. An example embodiment would be the collection of blood from a dog patient and the manual measurement of the White Blood Cells or Cholesterol within the blood sample. The output of the diagnostics test system is a set of diagnostics test results, for example, in an embodiment, comprising quantitative or qualitative assessment information based on a plurality of diagnostic dimensions.

### Result Interpretation System:

The result interpretation system 106 is designed to convert numerical veterinary test results, obtained from the previous system, into descriptive text summaries. In an embodiment, it employs a look-up table describing specific numerical ranges for each test result, in consideration for the specific animal, and converts the numerical tests into a written description of the patients results. For example, if a patient has a T4 reading of 150 and a Creatinine reading of 20, then the output of the result interpretation system would be “The patient has a high T4 and a low Creatinine.”. The interpretations can be more detailed than just high/medium/low and can include without limitation more specific ranges such as mildly high, higher side of normal, etc. In an embodiment, a rules-based approach is provided. In an embodiment, system comprises an interface to receive numerical veterinary test results comprising quantitative or qualitative assessment information determined from a biological sample, the quantitative or qualitative assessment information based on a plurality of diagnostic dimensions.

### Expert Database and Case Lookup System:

The text interpretations 107 of the previous system 106 are provided to system 108 where the text interpretations 107 are then compared to 1) a database (e.g. 108A) of expertly reviewed case files, and 2) a database (e.g. 108B) of similar cases with resulting reports. In this case, the first matching pass looks for an identical match (exam same result interpretations and animal history). If the first pass succeeds with an exact match, this is used in the next system. If the first pass fails, then a nearest neighbor algorithm is utilized to find the closest matching expert file (i.e. a case file where over T % of result parameters are the same, with typically a T value of 50+% being used in practice). If there is an identical or closest match, the case report along with the result interpretations are used in the subsequent stage. If there is no identical or closest match, then only the result interpretations are used in the next stage.

The closest match can be found in one of several ways. Disclosed herein is a system designed to identify and retrieve veterinary case records that exhibit a high degree of similarity to a newly presented case, referred to as the “exemplar.” The system leverages a series of weighted comparisons across a variety of clinically relevant attributes, which may include demographic data—such as age, breed, species, and gender—and laboratory or diagnostic data—such as T4 levels, total protein, creatinine, blood urea nitrogen, or liver enzymes. In addition to these core attributes, the system can also integrate clinical outcomes or treatment response data to refine its matching criteria over time. By systematically applying a weighted scoring mechanism, veterinary practitioners are enabled to rapidly locate previously documented cases that share crucial clinical features with the new case, thereby informing diagnosis and treatment strategies.

In a representative embodiment, each case in the repository is associated with a list of attribute-value pairs. For instance, one record might indicate that a particular cat is nine years old, neutered male, of the Siamese breed, and that its bloodwork shows a T4 value above the clinically recognized “normal” threshold, normal total protein, and slightly elevated liver enzymes. Another record might reflect a 12-year-old dog, female, with elevated total protein but no information about T4. When a new exemplar case is introduced, the system calculates a similarity score between that exemplar and every candidate case in the database by summing, for each attribute, the product of a specific weight and a similarity measure. In other words, for all the attributes considered, the system multiplies each attribute's weight by a value that indicates how well the exemplar matches that attribute in the candidate record, then adds those products together. A similarity measure might, for example, assign a value of 1.0 if both the exemplar and candidate have “High” T4 readings, and a value of zero if one reads “High” and the other “Normal.” The actual measure may be more nuanced, such as returning a fraction if the values are only slightly different, but the concept is to normalize the comparison so that it can be combined across attributes.

The selection and tuning of these attribute weights can have a large impact on results. In some scenarios, age may be regarded as only marginally important, receiving a lower weight (for example, 0.5 on a scale of zero to five), whereas the presence of high T4 might be more strongly weighted (for example, three or four), reflecting a belief that endocrine dysfunction is the main driver of a particular condition. A variety of methods can be employed for determining these weights. An experienced clinician might manually set them based on years of practice, adjusting them to emphasize factors most predictive of disease outcomes in cats versus dogs. Alternatively, a machine learning algorithm could be used to derive optimal weights from retrospective data. For instance, if the system continuously finds that cases highly ranked for T4 similarity correlate well with successful treatment plans, it might gradually increase the weight for T4. Conversely, if age differences do not correlate strongly with case outcomes, the algorithm might reduce the age weight over time. In a hybrid approach, a veterinarian might initialize a set of clinically sensible weights, and the system refines those weights based on an ongoing analysis of case records and outcomes.

A feature of the embodiment is its ability to handle situations where the exemplar's tests do not exactly match the set of tests found in the candidate database entries. Often in clinical practice—especially in veterinary care—different blood panels or diagnostic imaging results may be obtained, depending on the suspected disease, the client's financial constraints, or the clinic's protocols. Thus, an exemplar case might include a T4 value but lack any measurement of total protein, whereas a candidate case might have results for both T4 and total protein. The system therefore employs a flexible approach to partial comparisons. One option is to disregard attributes that do not overlap, basing the total similarity score only on the attributes both the exemplar and the candidate share. In other words, if the exemplar has T4 data but no total protein data, and the candidate has T4, total protein, and BUN, the similarity calculation focuses on T4 (and perhaps demographic attributes like breed or age), ignoring total protein or BUN comparisons.

Another strategy involves applying a minimal penalty for missing data. Instead of ignoring an attribute altogether, the system might subtract a small fraction of the weight for that attribute if the candidate record does not contain a measurement that the exemplar has. For example, if the weight for T4 is five and the candidate lacks T4 measurements, the system might subtract a fraction—such as 10% or 20%—of that weight from the overall similarity score. This approach prevents the absence of a particular test from being overlooked entirely while also ensuring that potentially relevant candidates remain in contention.

Additionally, if clinically appropriate proxy measures exist—such as free T4 instead of total T4—the system can implement a procedure to transform one metric into an approximation of the other. For example, if free T4 and total T4 readings are closely correlated in a particular species, the system may use a function to derive an approximate total T4 value from the free T4 measurement. This approach is especially valuable when older data uses one test methodology (such as total T4) while current patients are generally evaluated with the other (such as free T4), or when specialized tests become available partway through a longer data collection effort.

To illustrate the workflow, consider a scenario where the exemplar is a 10-year-old neutered male cat with a T4 reading (for instance, 6.0 micrograms per deciliter) that exceeds the normal reference range. Suppose no other blood tests were recorded for this cat. The system might query hundreds of cat records and yield three top candidates. The first is a nine-year-old neutered male cat with a T4 level of 5.8 micrograms per deciliter, normal total protein, and normal blood urea nitrogen. The second is a 12-year-old intact female cat whose T4 level is 6.2 micrograms per deciliter, with elevated total protein and no data for blood urea nitrogen. The third candidate matches the exemplar's age, gender, and breed precisely but lacks T4 data altogether. If T4 is highly weighted, the first two candidates would likely receive stronger similarity scores thanks to their high T4 readings matching the exemplar. However, the first candidate, having a nearly identical age and gender, might be granted a slightly higher overall match. The second might receive a moderate penalty for the mismatch in gender or for the elevated total protein, which is not measured in the exemplar (or not factored in if the system omits mismatched tests). The third candidate, lacking T4 data, could be penalized or might simply not gain any points for T4, depending on the chosen missing-data policy. Consequently, the system would likely rank the first candidate highest, the second candidate next, and the third candidate last, thereby ensuring that the cat most likely sharing the exemplar's clinically significant hyperthyroid indicator remains at the top.

In a second illustrative example, consider a hospital that treats a significant number of dogs with various degrees of kidney dysfunction. The veterinary staff might place more importance on creatinine and blood urea nitrogen measurements than on T4, since T4 is more commonly associated with hyperthyroidism in cats. In such a situation, an exemplar dog that presents with a blood urea nitrogen reading well above the normal reference range (for instance, 40 milligrams per deciliter when normal is 7 to 27 milligrams per deciliter) will match most closely with records that also indicate similarly high blood urea nitrogen levels. If half the records do not include blood urea nitrogen data because it was never tested, and the other half do, the system could penalize those missing the measurement by a small fraction of the blood urea nitrogen weight, ensuring that these partially complete records receive slightly lower overall similarity scores. Over time, the system might gather evidence showing that matching blood urea nitrogen readings does indeed correlate with better treatment guidance, leading to incremental increases in the weight for that attribute. Alternatively, if it turns out that blood urea nitrogen alignment does not consistently predict successful outcomes, the automated or hybrid tuning mechanism would gradually lower the attribute's weight.

By allowing both manual adjustment and algorithmic refinement, the embodiments accommodates differences in expert opinion, disease prevalence, and evolving medical standards. Its handling of missing or partially overlapping data ensures that relevant matches are not prematurely excluded, so cases lacking a single test but sharing the most significant clinical features remain in the pool of possible matches. Overall, the embodiments provide a more nuanced, adaptable, and clinically aligned approach to veterinary data retrieval compared to basic keyword or singular-attribute searches. The efficiency gained in identifying analogous cases can expedite diagnostic decisions, enhance prognoses, and potentially improve patient outcomes. Although examples have been provided to illustrate specific ways of weighting attributes and handling incomplete data, numerous modifications and variations can be practiced without departing from the spirit and scope of this disclosure.

In an embodiment, expert database and case lookup system 108 is configured to store and retrieve expert-reviewed case files, facilitating the matching of a current case represented by the descriptive text summary to similar cases in the database. In an embodiment, system 108 is configured to provide the similar cases (results 109) to a report generation system 114 (e.g. comprising systems 110 and 112) to generate a veterinary report using the similar cases.

### Veterinary-Specific Language Model System:

The results 109 of the previous system 108 which includes the result interpretations, details of the patient (including history and demographic information), as well as possibly the best matching expert file or case file, are then analyzed by a veterinary-specific language model system 110 having a veterinary-specific language model. Analysis which is comprised of:
1) Expanding veterinary specific language codes and names into a descriptive text based on a look-up table that matches keywords into expanded descriptions. For example, a mention of the medication “Thyronorm” is expanded to “Thyronorm (a medication used to lower T4 levels)”. Other veterinary-specific words are expanded with an explanation.
2) Removing unrelated or redundant verbiage and language which would not have significance in a veterinary context, based on a lookup table that includes or describes the context in which certain wording or phrases should be removed.

The result 111 of this system 110 comprises a draft veterinary report that includes the result interpretations, potentially matching similar case reports, and veterinary-specific expansions/removals.

### Large Language Model System:

The large language model (LLM) system 112 synthesizes the draft report (111) and incorporates it into the patient-specific context using a LLM. Leveraging its extensive knowledge and understanding of language, the large language model generates a comprehensive veterinary report e.g. as output 113.

The report 113 encapsulate a summary of the veterinary test results, interpretative insights, potential diagnoses, treatment recommendations, and further steps tailored to the individual patient.

The large language model has the specific goals of 1) cleaning up the language and grammar in the draft report and 2) cleaning up and confirming the diagnosis if one was made in the draft report, or generating a diagnosis if one was not already made.

In one embodiment, the Large Language Model can consist of a transformer-based language model trained and implemented as described below:

A transformer-based language model is a sophisticated neural network architecture designed to process sequential data-particularly text-by capturing contextual dependencies across entire sequences rather than only in a fixed window or order. When trained on a large veterinary database, such a model gains an extensive understanding of veterinary terminology, common diagnoses, treatments, patient demographics, laboratory values, and narrative report structures that professionals frequently use when documenting cases. Through its attention-based mechanisms, a transformer can integrate information from widely separated parts of a text, making it especially adept at capturing the wide array of clinical, diagnostic, and narrative details necessary for composing coherent, contextually accurate veterinary reports.

In a traditional transformer, there are multiple stacked layers that each feature a multi-headed self-attention component and a position-wise feed-forward network. The input to the transformer typically consists of tokenized text, where each token is represented by a numeric index corresponding to a vocabulary entry. An embedding layer converts these indices into dense vector representations, ensuring that semantically similar tokens have vectors that are close in their high-dimensional space. Alongside these embeddings, the transformer adds positional encodings that allow the network to understand the order of tokens. Because the transformer does not rely on recurrent connections or explicit directional processing, it needs a means of tracking sequential order, and positional encodings provide a sinusoidal or learned pattern that, once added to the embeddings, gives each position a unique identity.

The multi-headed attention mechanism is central to the transformer's power. In a single “head” of attention, the model splits the token embeddings into three parts called queries, keys, and values. The queries interact with the keys to create a set of attention scores, which effectively determine how much focus should be placed on each token in the sequence relative to the token under consideration. These scores are then applied to the values, producing a weighted sum that merges contextual information back into a single vector representation. By using multiple heads, the network can learn to attend to different aspects of the input simultaneously. One head might focus on anatomical terms, for instance, while another might focus on numerical lab results or breed-related risk factors. This multi-faceted attention process allows the transformer to track many relationships in parallel, which is invaluable for capturing nuanced medical knowledge.

Each attention sub-layer is followed by a position-wise feed-forward network that applies nonlinear transformations to each token's representation. The output of the feed-forward network is then combined with a “residual connection” from the attention sub-layer, and the combined result is normalized (layer normalization) to maintain stable gradients and improve training efficiency. These layers are stacked multiple times, allowing the model to progressively build up its representation of the input. The final output of these stacked layers can then be fed into a classification head (for tasks like classification) or a language modeling head (for generating text). In the context of writing veterinary reports, the model would typically be used in a language generation capacity, so it might be trained or fine-tuned to predict the next token in a sequence or to fill in missing tokens given a partial context.

The neural network training mechanism for a transformer-based model on a large veterinary database typically involves a two-step or multi-stage process. First, one might pre-train the model using a masked language modeling objective on an extensive corpus of veterinary records, textbooks, research articles, and de-identified clinical notes. This pre-training step exposes the model to an enormous variety of veterinary terminology, such as breed-specific ailments (e.g., hip dysplasia in large dog breeds), routine diagnostic procedures (blood chemistries, x-rays, ultrasound findings), standard treatments (antibiotic choices, surgical interventions), and narrative forms (clinic notes, discharge summaries). During masked language modeling, random tokens in the text are replaced with a special “[MASK]” symbol, and the model must learn to predict the missing tokens based on the context provided by surrounding tokens. This forces the transformer to pay close attention to context, gleaning domain-specific relationships among words and phrases. Over the course of this training, it learns that “canine hypothyroidism” is linked to certain lab findings (low T4, high TSH) and that “feline hyperthyroidism” usually corresponds to elevated T4 levels and potential concurrent conditions, for instance.

After acquiring this broad knowledge, the model might undergo fine-tuning for a specialized task: writing coherent veterinary reports. In this phase, the model is provided with example inputs that simulate the conditions under which a vet might need a completed report. For instance, the input might include a structured summary of the patient's signalment (species, breed, age, sex), presenting complaint, vital signs, laboratory results, any diagnostic imaging findings, and relevant clinical history. The training data includes not only the structured information but also a sample of high-quality veterinary reports written by experts. The model is then trained to produce fluent, contextually accurate text that integrates all the given clinical details into a narrative form. During fine-tuning, the network parameters are adjusted so that when it encounters a similar input in the future, it can compose an appropriate report.

To illustrate how the model would work in practice, consider a scenario in which a veterinarian has just completed an examination of a 4-year-old Golden Retriever presenting with intermittent diarrhea and mild weight loss. The veterinarian inputs the dog's demographics (female, intact, age 4 years), chief complaint (diarrhea for one week, decreased appetite), vital signs (temperature slightly elevated, heart rate within normal limits), and laboratory values (mild anemia, slightly increased white blood cell count, negative fecal exam for parasites). The model, pre-trained on general veterinary data and fine-tuned on writing consistent reports, processes these pieces of information token by token. Through the self-attention mechanism, it recalls related data about gastrointestinal diseases common to young dogs and the possible significance of mild anemia in a GI context. It might also integrate knowledge of typical results for fecal tests and the significance of a negative parasite screen.

When the veterinarian requests a written report, the transformer-based system generates a coherent paragraph that may begin as follows: “Patient presented is a 4-year-old intact female Golden Retriever with a primary complaint of diarrhea ongoing for seven days and a mild decrease in appetite. Physical examination revealed a slightly elevated rectal temperature, while heart rate remained within normal ranges. A complete blood count indicated mild anemia and a moderately increased leukocyte count. Fecal analysis was negative for common parasitic organisms.” From there, it can continue to propose an assessment: “Clinical signs, in conjunction with bloodwork results, suggest a possible inflammatory or infectious etiology, though the absence of parasites on fecal exam makes helminthic causes less likely.” The system might then summarize a recommendation for further diagnostics: “Given the persistence of gastrointestinal signs, additional tests such as abdominal ultrasound or serum chemistry for pancreatic evaluation may be warranted.”

Throughout this process, the model relies on its training to balance clarity, conciseness, and completeness. It organizes its output in a manner similar to what is seen in real veterinary medical records, with an opening statement of the reason for the visit, then an interpretive section describing findings, and finally a recommendation or conclusion. When the veterinarian or a clinic staff member reviews and edits the generated text, they can correct any factual errors (if the model misunderstood or improperly integrated some data) or augment it with more specific clinical insights, effectively collaborating with the model to produce a polished final report.

As a further example, the same system could adapt to writing a discharge summary. If a cat was hospitalized for postoperative care following a femoral fracture repair, the system would glean from the input data that the cat's procedure took place on a certain date, that the hardware used for fixation was standard intramedullary pins and orthopedic screws, and that the cat was to be discharged on a pain management regimen. The generated text might read: “This 2-year-old male neutered Domestic Shorthair cat is recovering from femoral fracture repair performed on August 3rd. The surgical site has been stable upon daily inspection, with no signs of infection such as redness, swelling, or discharge. The patient has been receiving opioid analgesics for pain control, which will continue at home for five days. Activity should be restricted to a contained area, and follow-up radiographs are recommended in six weeks to confirm bone healing progress.”

By employing a comprehensive transformer-based language model, veterinary professionals can streamline documentation, especially in busy clinical settings or high-throughput research environments. The model's ability to integrate domain-specific knowledge with general language fluency means that the reports it produces usually read naturally and adhere to professional standards. Moreover, because the underlying neural network captures patterns from large corpora of cases, it can often include subtle but important details-like instructions for medication tapering or watch-outs for post-surgical complications-without needing frequent manual insertion of boilerplate text. Although a human veterinarian remains essential for validating accuracy and providing final oversight, the time and cognitive load spent in writing extensive reports are substantially reduced, leading to improved efficiency and patient record consistency.

### Operation:

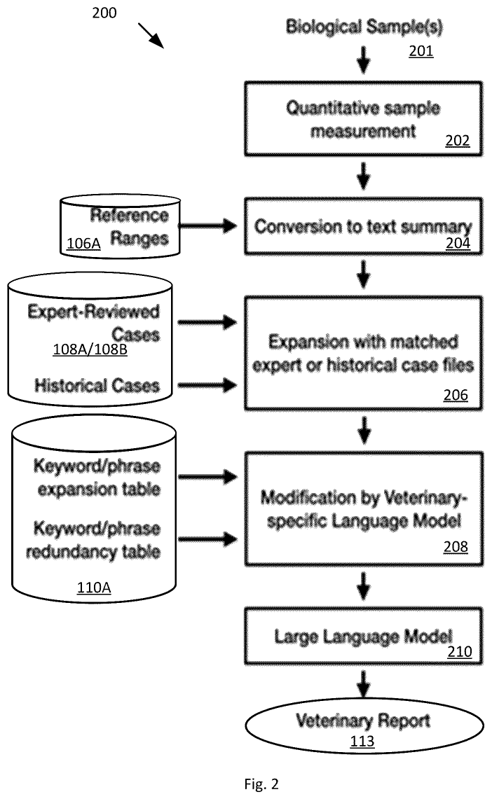

Operation of system 100, in accordance with an embodiment, will be understood with reference to FIG. 2 illustrating a flow chart 200 having steps 202, 204, 206, 208 and 210. Further shown are inputs from data stores 106A, 108A, and 110A, along with output 113.

After obtaining a biological sample 201 and generating (at 202) a numerical veterinary test results 103 as input, the result interpretation system 106 converts (e.g. at 204) this data (using 106A) into descriptive text summaries 107.

These summaries 107 are used at 206 to find relevant expert or historical case files (from 108A) and the combination of the matching files (if any) and the summaries are then fed into the veterinary-specific language model system 110, which (at 208) searches and retrieves relevant expert reports from the database 110A to create a draft report 111.

Finally, the large language model system 112 regenerates at 210 the draft report 111 to generate a final personalized veterinary report 113 summarizing the veterinary test results and recommending appropriate next steps.

There is provided, in an embodiment, a method for the generation of a veterinary report, comprising: A) receiving of one or more biological samples; B) performing an evaluation of the one or more biological samples based on a plurality of veterinary test dimensions to produce numerical test results; C) utilizing a result interpretation system to convert the numerical test results into a descriptive text summary; D) employing a database and case lookup system to search and retrieve relevant expert reports from an expert database based on the descriptive text summary; E) utilizing a veterinary-specific language model to expand key veterinary terms and remove redundant information to prepare a draft report for the veterinary report; and F) utilizing a large language model to regenerate a final personalized reports as the veterinary report that summarizes the test results and recommends appropriate next steps.

### EXAMPLES

To illustrate features herein, consider the case of a canine blood test. A blood sample is collected from the patient and the series of test dimensions are analyzed (such as white blood cells, cholesterol, etc.). The result interpretation system processes the numerical values, converting them into descriptive summaries. These summaries are used to retrieve relevant expert or historical case reports from the database, integrating them with the interpretation system's output. The veterinary-specific language model then expands the draft report with additional information and context and removes redundant information. The large language model synthesizes this information to generate a comprehensive report including potential diagnoses, treatment recommendations, and additional steps specific to the canine patient.

### Statements

Various features and aspects of the present disclosure include those in the following enumerated Statements:
Statement 1: A system, comprising: A) an interface to receive numerical veterinary test results comprising quantitative or qualitative assessment information determined from a biological sample, the quantitative or qualitative assessment information based on a plurality of diagnostic dimensions; B) a result interpretation system to convert the numerical veterinary test results into a descriptive text summary using algorithms for analysis and interpretation; C) an expert database and case lookup system to store and retrieve expert-reviewed case files, facilitating the matching of a current case represented by the descriptive text summary to similar cases in the database; D) a report generation system to generate a veterinary report using the similar cases.
Statement 2: The system of Statement 1, further comprising a veterinary-specific language model that utilizes look up tables to expand key veterinary definitions and to remove redundant information to prepare a draft report for generating the veterinary report.
Statement 3: The system of Statement 2, wherein the veterinary-specific language model employs a similarity-based retrieval mechanism to identify and retrieve expert-generated reports aligned with the analyzed veterinary test results, integrating them with the result interpretation system to generate the draft report.
Statement 4: The system of Statement 2, further comprising a large language model that synthesizes the draft report, incorporating it into patient-specific context, and generating the veterinary report to encapsulate a summary of the test results, interpretative insights, potential diagnoses, treatment recommendations, and further steps tailored to individual patients.
Statement 5: The system of Statement 1 comprising one or more sample testing devices to analyze the biological sample and provide the numerical veterinary test results to the interface.
Statement 6: A method for the generation of a veterinary report, comprising: A) receiving of one or more biological samples; B) performing an evaluation of the one or more biological samples based on a plurality of veterinary test dimensions to produce numerical test results; C) utilizing a result interpretation system to convert the numerical test results into a descriptive text summary; D) employing a database and case lookup system to search and retrieve relevant expert reports from an expert database based on the descriptive text summary; E) utilizing a veterinary-specific language model to expand key veterinary terms and remove redundant information to prepare a draft report for the veterinary report; and F) utilizing a large language model to regenerate a final personalized reports as the veterinary report that summarizes the test results and recommends appropriate next steps.
Statement 7: The method of Statement 6, wherein the result interpretation system employs algorithms for pattern recognition and clinical guidelines to analyze and interpret the significance of individual test results of the numerical test results.
Statement 8: The method of Statement 6, wherein the veterinary-specific language model uses similarity-based retrieval to identify and retrieve expert-generated reports aligned with the descriptive text summary.
Statement 9: The method of Statement 6, wherein the large language model leverages extensive knowledge and understanding of language to synthesize the draft report and incorporate it into the patient-specific context.
Statement 10: A computer-implemented method for retrieving veterinary case records based on similarity to an exemplar case, the method comprising: receiving, via an input interface, one or more attributes of the exemplar case, wherein the attributes include at least one demographic attribute and at least one diagnostic attribute; retrieving, from a data repository, a plurality of candidate veterinary case records, each record comprising stored demographic attributes and stored diagnostic attributes; for each candidate veterinary case record, comparing the exemplar case attributes to the candidate record's corresponding attributes by generating, for each attribute in a set of overlapping attributes, a similarity measure indicative of how closely the candidate record matches the exemplar case; applying a weight to each similarity measure, wherein the weight is selected to reflect the relative importance of that attribute in determining case similarity; combining the weighted similarity measures to compute a final similarity score for each candidate veterinary case record; and ranking the plurality of candidate veterinary case records in order of descending similarity score and transmitting, via an output interface, an indication of one or more top-ranked candidate veterinary case records.
Statement 11: The method of Statement 10, wherein generating the similarity measure for an attribute comprises: determining whether the exemplar case attribute and the candidate record attribute each exceed or fall below a predefined clinical threshold, and assigning a maximum similarity value if both attributes lie on the same side of the threshold, or assigning a reduced similarity value if they do not.
Statement 12: The method of Statement 10, further comprising, prior to combining the weighted similarity measures, determining that the candidate veterinary case record lacks one or more attributes measured in the exemplar case, and adjusting the candidate record's overall similarity score to account for the absence of said attributes.
Statement 13: The method of Statement 10, further comprising dynamically tuning one or more of the weights by at least one of: (a) manual adjustment by a veterinary practitioner based on clinical judgment; (b) automated adjustment using historical case outcomes and a feedback model; or (c) a hybrid approach that initiates with practitioner-assigned values and refines said values using data-driven analysis.
Statement 14: The method of Statement 10, wherein combining the weighted similarity measures comprises adding products of the form “attribute weight times normalized similarity measure” for all overlapping attributes in the exemplar case and the candidate record, such that each candidate record receives a single final similarity score.
Statement 15: A system for retrieving veterinary case records based on similarity to an exemplar case, the system comprising: a data repository storing a plurality of veterinary case records, each having a set of demographic attributes and a set of diagnostic attributes; an input interface configured to receive one or more demographic and diagnostic attributes of the exemplar case; a processing module configured to:-compare the exemplar case attributes to each of the veterinary case records in the data repository, including generating, for each overlapping attribute, a similarity measure between the exemplar case and the respective veterinary case record;-apply a plurality of weights to the respective similarity measures, each weight assigned to a particular attribute or group of attributes;-calculate a final similarity score for each veterinary case record based on the weighted similarity measures; and-rank the veterinary case records in an order of descending final similarity score; and an output interface configured to provide an indication of the ranked veterinary case records to a user.
Statement 16: The system of Statement 15, wherein the data repository stores a threshold-based classification for each diagnostic attribute, and the processing module is further configured to compare any diagnostic attribute in the exemplar case that is classified as “High,” “Low,” or “Normal” with the corresponding classification in a candidate record to determine the similarity measure.
Statement 17: The system of Statement 15, wherein the processing module is further configured to handle non-overlapping attributes by performing at least one of: (a) omitting non-overlapping attributes from the similarity score calculation; (b) assigning a penalty for missing attributes in the candidate record; or (c) mapping a proxy attribute in the candidate record to the exemplar case attribute based on a predetermined correlation.
Statement 18: The system of Statement 15, wherein the processing module further applies a feedback process, in which an outcome metric associated with at least one veterinary case record is used to modify the weights assigned to one or more attributes, thereby improving future matching accuracy.
Statement 19: A non-transitory computer-readable medium having stored thereon instructions that, when executed by one or more processors, cause a system to: receive an exemplar case comprising one or more demographic attributes and one or more diagnostic attributes; retrieve a plurality of veterinary case records, each including demographics and diagnostics; for each veterinary case record, generate a partial similarity measure for each overlapping attribute and apply an attribute-specific weight to form a weighted similarity value; aggregate the weighted similarity values to obtain a final similarity score for the veterinary case record; handle missing attributes in the veterinary case record by either assigning a penalty or excluding the missing attribute comparison; and return a ranked list of veterinary case records, sorted from highest to lowest final similarity score, for display to a user.

### Interpretation:

Any computing device herein can be implement using one or more processors and storage device(s) storing instructions that, when executed, cause the computing device to perform operations. Examples of hardware include a general-purpose central processing unit (CPU) programmed using software, a graphical processing unit (GPU) also as programmed or other processor, microprocessor, or microcontroller options having sufficient resources and capacity.

It is understood that other types of circuitry than programmable processors can be configured. Hardware components comprising specifically designed circuits can be employed such as but not limited to an application specific integrated circuit (ASIC) or other hardware designed to perform specific functions, which may be more efficient in comparison to a general-purpose CPU programmed using software. Thus, broadly herein an apparatus aspect relates to a system or device having circuitry (sometimes referenced as computational circuitry) that is configured to perform certain operations described herein, such as, but not limited, to those of a method aspect herein, whether the circuitry is configured via programming or via its hardware design.

Practical implementation may include any or all the features described herein. These and other aspects, features and various combinations may be expressed as methods, apparatus, systems, means for performing functions, program products, and in other ways, combining the features described herein. Several embodiments have been described. Nevertheless, it will be understood that various modifications can be made without departing from the spirit and scope of the processes and techniques described herein. In addition, other steps can be provided, or steps can be eliminated, from the described process, and other components can be added to, or removed from, the described systems. Accordingly, other embodiments are within the scope of the following claims.

Throughout the description and claims of this specification, the word “comprises” and “contain” and variations of them mean “including but not limited to” and they are not intended to (and do not) exclude other components, integers or steps. Throughout this specification, the singular encompasses the plural unless the context requires otherwise. Where the indefinite article is used, the specification is to be understood as contemplating plurality as well as singularity, unless the context requires otherwise.

Features, integers characteristics, compounds, chemical moieties or groups described in conjunction with a particular aspect, embodiment or example of the invention are to be understood to be applicable to any other aspect, embodiment or example unless incompatible therewith. All the features disclosed herein (including any accompanying claims, abstract and drawings), and/or all the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings) or to any novel one, or any novel combination, of the steps of any method or process disclosed.

### CONCLUSION

The automated veterinary report generator described herein revolutionizes the interpretation of veterinary test results, providing accurate and personalized reports. The integration of result interpretation, expert database, and language models ensures a comprehensive understanding of each case, enhancing the quality of veterinary care. The embodiments have broad applications in veterinary clinics, research institutions, and other settings where precise interpretation of test results is critical to effective animal healthcare.

1. A system comprising at least one processor and at least one storage device storing instructions executable by the at least one processor to provide:
A) an interface to receive numerical veterinary test results comprising quantitative or qualitative assessment information determined from a biological sample, the quantitative or qualitative assessment information based on a plurality of diagnostic dimensions;
B) a result interpretation system to convert the numerical veterinary test results into a descriptive text summary using algorithms for analysis and interpretation;
C) an expert database and case lookup system to store and retrieve expert-reviewed case files, facilitating the matching of a current case represented by the descriptive text summary to similar cases in the database; and
D) a report generation system to generate a veterinary report using the similar cases.

2. The system of claim 1, further comprising a veterinary-specific language model that utilizes look up tables to expand key veterinary definitions and to remove redundant information to prepare a draft report for generating the veterinary report.

3. The system of claim 2, wherein the veterinary-specific language model employs a similarity-based retrieval mechanism to identify and retrieve expert-generated reports aligned with the analyzed veterinary test results, integrating them with the result interpretation system to generate the draft report.

4. The system of claim 2, further comprising a large language model that synthesizes the draft report, incorporating it into patient-specific context, and generating the veterinary report to encapsulate a summary of the test results, interpretative insights, potential diagnoses, treatment recommendations, and further steps tailored to individual patients.

5. The system of claim 1 comprising one or more sample testing devices to analyze the biological sample and provide the numerical veterinary test results to the interface.

6. A method for the generation of a veterinary report, comprising:
A) receiving of one or more biological samples;
B) performing an evaluation of the one or more biological samples based on a plurality of veterinary test dimensions to produce numerical test results;
C) utilizing a result interpretation system to convert the numerical test results into a descriptive text summary;
D) employing a database and case lookup system to search and retrieve relevant expert reports from an expert database based on the descriptive text summary;
E) utilizing a veterinary-specific language model to expand key veterinary terms and remove redundant information to prepare a draft report for the veterinary report; and
F) utilizing a large language model to regenerate a final personalized reports as the veterinary report that summarizes the test results and recommends appropriate next steps.

7. The method of claim 6, wherein the result interpretation system employs algorithms for pattern recognition and clinical guidelines to analyze and interpret the significance of individual test results of the numerical test results.

8. The method of claim 6, wherein the veterinary-specific language model uses similarity-based retrieval to identify and retrieve expert-generated reports aligned with the descriptive text summary.

9. The method of claim 6, wherein the large language model leverages extensive knowledge and understanding of language to synthesize the draft report and incorporate it into the patient-specific context.

10. A computer-implemented method for retrieving veterinary case records based on similarity to an exemplar case, the method comprising:
receiving, via an input interface, one or more attributes of the exemplar case, wherein the attributes include at least one demographic attribute and at least one diagnostic attribute;
retrieving, from a data repository, a plurality of candidate veterinary case records, each record comprising stored demographic attributes and stored diagnostic attributes;
for each candidate veterinary case record, comparing the exemplar case attributes to the candidate record's corresponding attributes by generating, for each attribute in a set of overlapping attributes, a similarity measure indicative of how closely the candidate record matches the exemplar case;
applying a weight to each similarity measure, wherein the weight is selected to reflect the relative importance of that attribute in determining case similarity;
combining the weighted similarity measures to compute a final similarity score for each candidate veterinary case record; and
ranking the plurality of candidate veterinary case records in order of descending similarity score and transmitting, via an output interface, an indication of one or more top-ranked candidate veterinary case records.

11. The method of claim 10, wherein generating the similarity measure for an attribute comprises:
determining whether the exemplar case attribute and the candidate record attribute each exceed or fall below a predefined clinical threshold, and assigning a maximum similarity value if both attributes lie on the same side of the threshold, or assigning a reduced similarity value if they do not.

12. The method of claim 10, further comprising, prior to combining the weighted similarity measures, determining that the candidate veterinary case record lacks one or more attributes measured in the exemplar case, and adjusting the candidate record's overall similarity score to account for the absence of said attributes.

13. The method of claim 10, further comprising dynamically tuning one or more of the weights by at least one of:
(a) manual adjustment by a veterinary practitioner based on clinical judgment;
(b) automated adjustment using historical case outcomes and a feedback model; or
(c) a hybrid approach that initiates with practitioner-assigned values and refines said values using data-driven analysis.

14. The method of claim 10, wherein combining the weighted similarity measures comprises adding products of the form “attribute weight times normalized similarity measure” for all overlapping attributes in the exemplar case and the candidate record, such that each candidate record receives a single final similarity score.

15. The system of claim 1 further configured for retrieving veterinary case records based on similarity to an exemplar case, the system comprising:
a data repository storing a plurality of veterinary case records, each having a set of demographic attributes and a set of diagnostic attributes;
an input interface configured to receive one or more demographic and diagnostic attributes of the exemplar case;
instructions executable by the at least one processor configure the system to:
compare the exemplar case attributes to each of the veterinary case records in the data repository, including generating, for each overlapping attribute, a similarity measure between the exemplar case and the respective veterinary case record;
apply a plurality of weights to the respective similarity measures, each weight assigned to a particular attribute or group of attributes;
calculate a final similarity score for each veterinary case record based on the weighted similarity measures; and
rank the veterinary case records in an order of descending final similarity score; and
an output interface configured to provide an indication of the ranked veterinary case records to a user.

16. The system of claim 15, wherein the data repository stores a threshold-based classification for each diagnostic attribute, and the instructions executable by the at least one processor configure the system to compare any diagnostic attribute in the exemplar case that is classified as “High,” “Low,” or “Normal” with the corresponding classification in a candidate record to determine the similarity measure.

17. The system of claim 15, wherein the instructions executable by the at least one processor configure the system to handle non-overlapping attributes by performing at least one of:
(a) omitting non-overlapping attributes from the similarity score calculation;
(b) assigning a penalty for missing attributes in the candidate record; or
(c) mapping a proxy attribute in the candidate record to the exemplar case attribute based on a predetermined correlation.

18. The system of claim 15, wherein the instructions executable by the at least one processor configure the system to further apply a feedback process, in which an outcome metric associated with at least one veterinary case record is used to modify the weights assigned to one or more attributes, thereby improving future matching accuracy.

19. The system of claim 1, wherein the instructions executable by the at least one processor configure the system to:
receive an exemplar case comprising one or more demographic attributes and one or more diagnostic attributes;
retrieve a plurality of veterinary case records, each including demographics and diagnostics;
for each veterinary case record, generate a partial similarity measure for each overlapping attribute and apply an attribute-specific weight to form a weighted similarity value;
aggregate the weighted similarity values to obtain a final similarity score for the veterinary case record;
handle missing attributes in the veterinary case record by either assigning a penalty or excluding the missing attribute comparison; and
return a ranked list of veterinary case records, sorted from highest to lowest final similarity score, for display to a user.
